(19) United States
(12) Patent Application Publication
(10) Pub. No.: US 2010/0087712 A1
(43) Pub. Date: Apr. 8, 2010

(54) ENDOSCOPE

(75) Inventor: Hiroshi ITO, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS CORPORATION, Tokyo (JP)

(21) Appl. No.: 12/573,488

(22) Filed: Oct. 5, 2009

(30) Foreign Application Priority Data
Oct. 8, 2008 (JP) JP 2008-261736

(51) Int. Cl. A61B 1/06

(52) U.S. Cl. 600/160

(57) ABSTRACT

An endoscope according to the present invention has an insertion portion and characterized by comprising an electronic component housed in an end portion of the insertion portion and a Joule-Thomson cooling apparatus provided in a tube of the endoscope to cool the electronic component.

### CROSS-REFERENCE TO RELATED APPLICATION

The present application is based upon and claims the benefit of priority from the prior Japanese Patent Application No. 2008-261736 filed on Oct. 8, 2008; the entire contents of which are incorporated herein by reference.

### BACKGROUND OF THE INVENTION

1. Field of the Invention

The present invention relates to an endoscope equipped with a Joule-Thomson cooling apparatus for cooling an electronic component housed in the distal end portion of the endoscope.

2. Description of the Related Art

In the field of imaging using an endoscope, an imaging technique called special illumination imaging, which is different from conventional imaging using white light illumination, has become widely used as a technique that enables easy detection of lesions. Examples of the special illumination imaging include narrow band imaging (NBI) and auto fluorescence imaging (AFI). In the special illumination imaging, a part of the wavelength of light is used for imaging, and therefore an image pickup element having a high sensitivity is needed. In particular, in the AFI, since light used is not reflected light but weak fluorescence, an image pickup element having a higher sensitivity is needed. In order to achieve good image quality in imaging with weak light, it is effective to cool the imaging element to thereby reduce dark current.

With development of an image pickup module having higher functionality, higher density and LED (light emitting diode) illumination provided in the distal end portion of an endoscope, the quantity of heat generated in the distal end portion of the endoscope tends to increase. This causes an increase in the temperature of the image pickup element used therein, which leads to deterioration of image quality. To achieve good image quality, it is necessary to dissipate heat from the distal end portion of the endoscope or cool the distal end portion.

For example, in a known technique using a fluid for heat dissipation, a fluid channel through which a fluid flows is provided in an endoscope equipped with a light emitting diode unit (which will be hereinafter referred to as an LED unit) for illumination to carry away heat from the LED unit, as disclosed in Japanese Patent Application Laid-Open No. 2003-38437.

This method will be described with reference to FIG. 7. FIG. 7 is a schematic block diagram of a conventional endoscope 502.

The endoscope 502 has a light source portion 520 having an LED 534, a pump 542, a tank 523 in which water W is stored, pipes 541A, 541B, 545A, 545B and an intermediate pipe 546 that form a circulation conduit, and a temperature sensor 532. The temperature of the light source portion 520, which generates heat, provided in the operation portion of the endoscope 502 is measured by the temperature sensor 532. If the temperature measured is higher than a specific temperature, a pump drive control circuit 544 causes the pump 542 to operate. In consequence, water W flows or circulates continuously in the circulation conduit formed by the pipe 541A, pipe 545A, intermediate pipe 546, pipe 545B, and pipe 541A to cool the light source portion 520. The cooling water W absorbs heat from the light source portion 520, which is a heat source, to cool it as it flows in the circulation conduit.

In cases where the LED and other components are disposed in the operation portion as is the case with the above-described endoscope 502, cooling may be achieved by circulating water W. However, in cases where an image pickup module having higher functionality, higher density and LED (light emitting diode) illumination is used in the distal end portion of the insert portion of the endoscope, satisfactory heat dissipation or cooling performance cannot be achieved only by circulating water W. Water W may be cooled before or after supplied by the pump 542. However, since the diameter of the insert portion of the endoscope 502 is small, the allowable diameter of the fluid channel through which water W flows is small, and the length of the fluid channel is relatively long. In consequence, the quantity of water W supplied is very small, and the temperature of water W will rise to become equal to the environmental temperature or the human body temperature as water W slowly flows in the narrow, long channel in the insert portion. Therefore, it is difficult to cool (or dissipate heat from) the electronic components such as the LED.

### SUMMARY OF THE INVENTION

The present invention has been made in view of the above described problem. An object of the present invention is to provide an endoscope having an insert portion equipped with a cooling system that can cool an electronic component(s) such as an image pickup element and/or an LED housed in the distal end portion of the insert portion, in particular, a system that can cool the electronic component to a temperature lower than the temperature of the environment in which the endoscope is used.

According to the present invention, there is provided an endoscope comprising an insertion portion, an electronic component housed in the distal end portion of the insertion portion, a Joule-Thomson cooling apparatus (which will be sometimes referred to as a “JT cooling apparatus” hereinafter) provided in a tube of the endoscope to cool the electronic component.

According to a preferred mode of the present invention, it is desirable that the Joule-Thomson cooling apparatus comprise a straight double tube.

According to a preferred mode of the present invention, it is desirable that the double tube be flexible.

According to a preferred mode of the present invention, it is desirable that the Joule-Thomson cooling apparatus comprise a very thin tube that extends at least in a bending portion of the endoscope and functions as a depressurization portion.

According to a preferred mode of the present invention, it is desirable that wherein the length of the depressurization portion be longer than the bending portion of the endoscope, the depressurization portion extend along the entire length of the bending portion of the endoscope portion, and the depressurization portion be made of a resin material.

According to a preferred mode of the present invention, it is desirable that an inner tube and an outer tube that constitute the double tube be both made of a resin material, and the depressurization portion be located in an end chip.

In the context of this specification, cooling an electronic component means dissipating heat generated from the electronic part or depriving the electronic part of heat.

### BRIEF DESCRIPTION OF THE DRAWINGS

FIG. 1 is a diagram showing the overall configuration of an endoscope system;

FIG. 2 is a cross sectional view of a distal end portion of an endoscope according to a first embodiment;

FIG. 3 is a perspective view of a JT cooling apparatus;

FIG. 4 is a cross sectional view taken on plane ABC in FIG. 3;

FIG. 5 is a cross sectional view showing the positional relationship of a rigid portion, a bending portion, and a flexible portion with a heat exchanger, a depressurization portion, and an end chip of the JT cooling apparatus;

FIG. 6 is across sectional view of a JT cooling apparatus according to a second embodiment, in which a pressurization portion is located in an end chip; and

FIG. 7 is a schematic block diagram of a conventional endoscope.

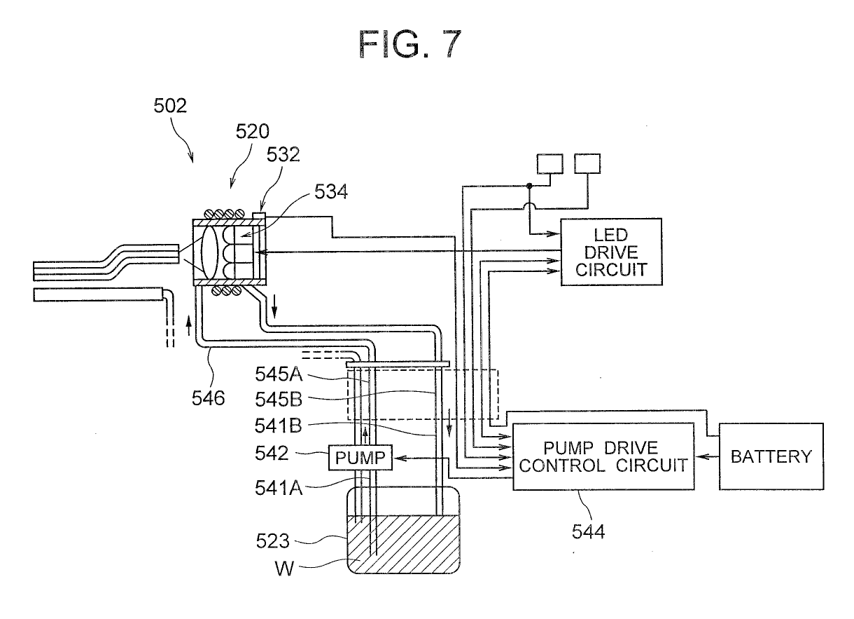

### DETAILED DESCRIPTION OF THE INVENTION

In the following, an embodiment of the endoscope according to the present invention will be described in detail with reference to the drawings. It should be understood that the present invention is not limited to the embodiments. To facilitate understanding of the configuration, hatching is omitted on some cross sections in the drawings.

### First Embodiment

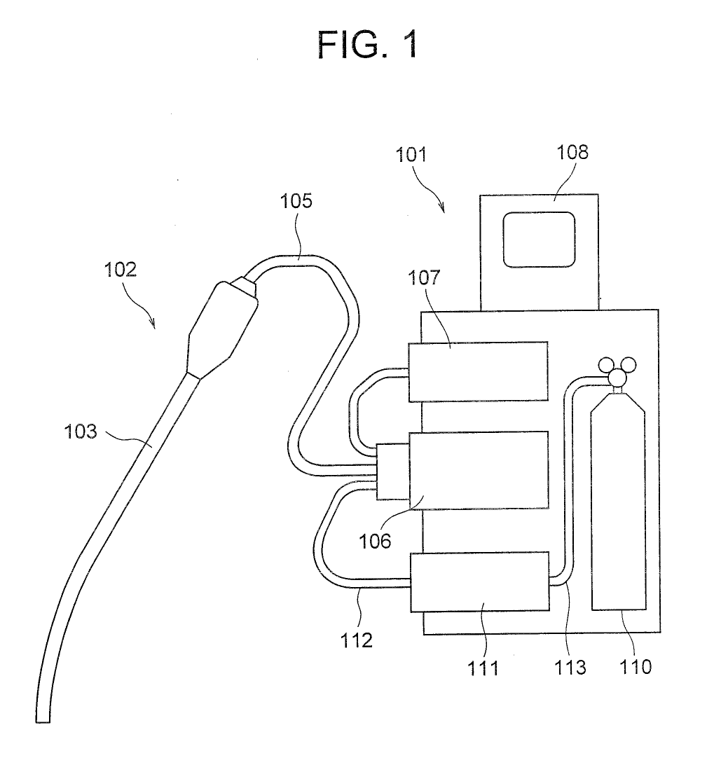

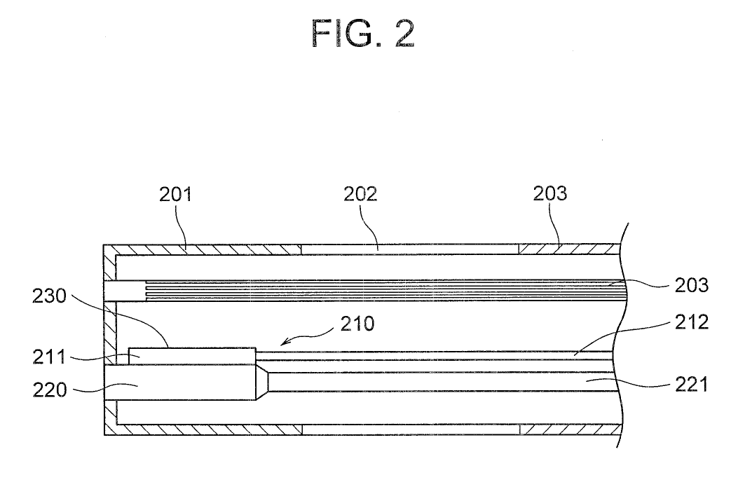

Here, a case where an endoscope according to a first embodiment of the present invention is built in an endoscope system 101 will be described by way of example. FIG. 1 is a diagram showing the overall configuration of the endoscope system 101. FIG. 2 is a cross sectional view of the distal end portion of the endoscope 102 according to the first embodiment.

The endoscope system 101 is mainly composed of the endoscope 102, a video processor 106, a light source 107, a monitor 108, a refrigerant gas controller 111, and a refrigerant gas cylinder 110. The refrigerant gas cylinder 110 is connected with the refrigerant gas controller 111 through a pipe 113, and the flow rate of the refrigerant gas is regulated by the refrigerant gas controller 111 in such a way that the temperature of the end chip 211 is kept constant at a certain temperature, whereby the refrigerant gas has an appropriate high pressure. The refrigerant gas regulated by the refrigerant gas controller 111 is supplied to a Joule-Thomson (JT) cooling apparatus 210 provided in the endoscope 102 through a pipe 112, and a pipe (not shown) provided in the interior of the video processor 106 and a universal cable 105.

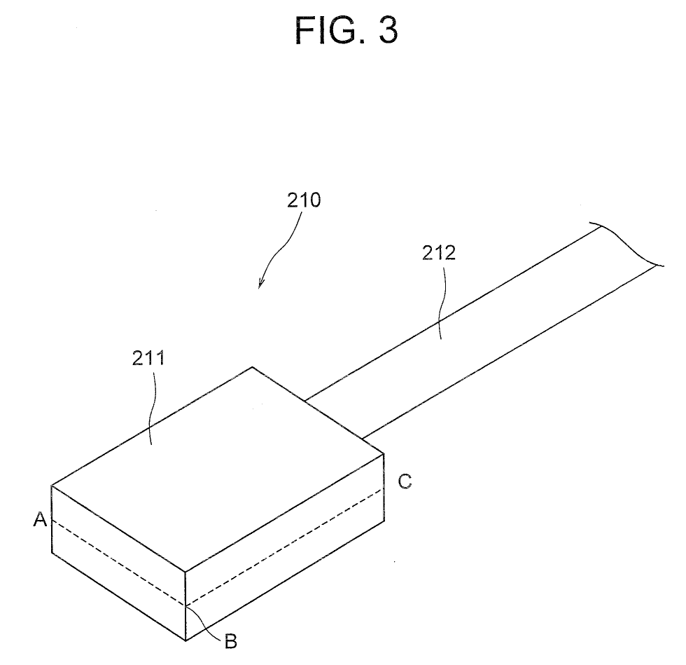

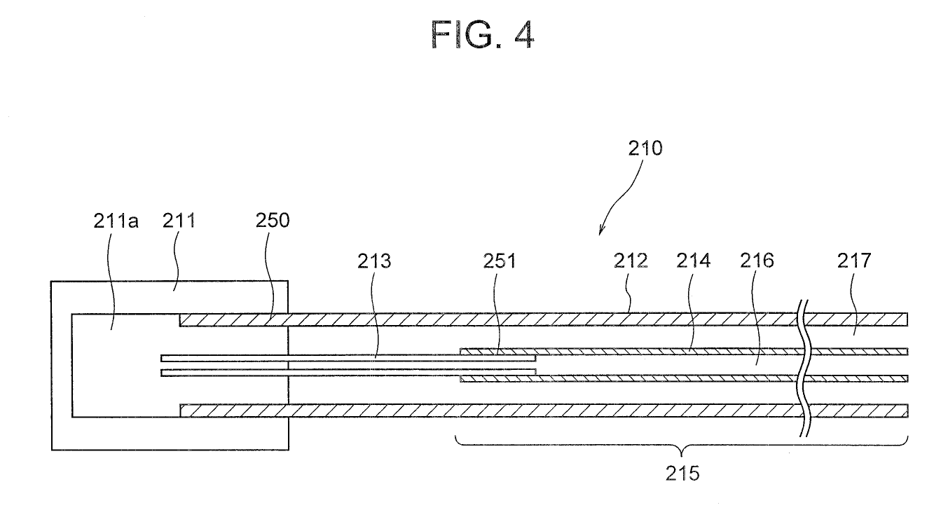

FIG. 3 is a perspective view of the JT cooling apparatus 210. FIG. 4 is a cross sectional view taken on plane ABC in FIG. 3. The JT cooling apparatus serving as heat dissipating/cooling means includes a heat exchanger 215 (composed of an inner tube 214 and an outer tube 212), a depressurization portion 213, and an end chip 211. The inner tube 214 and the outer tube 212 are flexible tubes with small diameters, which are disposed substantially concentric with each other to constitute a straight double tube. The inner tube 214 and the outer tube 212 extend in the insert portion 103 of the endoscope 102, and the inner tube 214 is connected to a pipe (not shown) for supplying refrigerant gas at a normal temperature and a high pressure provided in the universal cable 105.

When the outer tube 212 and the inner tube 214 are connected to pipes in the universal cable 105, the outer tube 212 and the inner tube 214 that form a double tube structure are separated into two separate tubes for connection. The depressurization portion 213 is a flexible tube having a very small diameter. The inner diameter of the depressurization portion 213 is smaller than that of the inner tube 214. The depressurization portion 213 is airtightly connected with the connection portion 251 of the inner tube 214. The end chip 211 has, in its interior, a cavity 211a in which the refrigerant gas flows. The end chip 211 is airtightly connected with a connection portion 250 of the outer tube 212.

The end chip 211 is adapted to be in contact with an image pickup module 220, which is the electronic component to be cooled, via thermally conductive grease or the like, which makes the thermal resistance between the end chip 211 and the image pickup module 220 becomes low. As the refrigerant gas flows in the cavity 211a of the end chip 211, it absorbs heat generated by the image pickup module 220 through the end chip 211. It is preferred that the end chip 211 be made of a material having a high heat conductivity. Furthermore, it is preferred that the side 230 of the end chip 211 that is not in contact with the object to be cooled (that is, the image pickup module 220, in this embodiment) is thermally insulated so that heat absorbed by the JT cooling apparatus 210 is limited, as much as possible, to the heat generated by the object to be cooled.

Although FIG. 2 shows a case in which the end chip 211 cools the image pickup module 220, the end chip 211 may be adapted to cool an LED module in the case of an endoscope using the LED illumination.

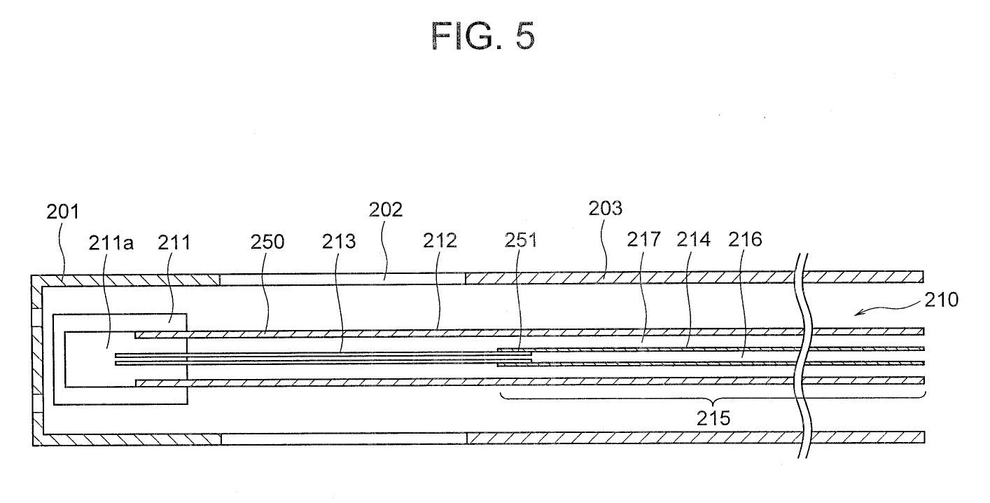

FIG. 5 is a cross sectional view showing the positional relationship of a rigid portion 201, a bending portion 202, and a flexible portion 203 with the heat exchanger 215, the depressurization portion 213, and the end chip 211 of the JT cooling apparatus 210. Illustration of the image pickup module and the light guide is omitted in FIG. 5.

The end chip 211 is located in the rigid portion 201, and the heat exchanger 215 is located in the flexible portion 203. The depressurization portion 213 extends all along the bending portion 202, and the two ends of the depressurization portion 213 are located respectively in the rigid portion 201 and the flexible portion 203.

The material of the inner tube 214 of the heat exchanger 215 disposed in the flexible portion 203 is a metal or a resin that has flexibility that the flexible portion 203 needs to have. Examples of such a resin include PEEK (registered trademark of Victrex: Poly Ether Ether Ketone resin), polyimide, and polyurethane. To improve the heat exchange efficiency between the channel 216 and the channel 217, it is preferred that the material of the inner tube 214 be a metal. For example, a tube made of SUS304 having an inner diameter of 0.3 mm, and outer diameter of 0.4 mm is as flexible as the flexible portion 203 needs to be. Since it is desirable that the interior and the exterior of the outer tube 212 be thermally insulated from each other, it is preferred that the outer tube 212 be made of a resin material having a low thermal conductivity. The heat exchanger 215 is designed to have a significant length to enhance the heat exchange efficiency.

It is preferred that the depressurization portion 213 extend along the entire length of the bending portion 202 and be made of a resin material. The aforementioned resin materials for the outer tube 212 may also be used as the material of the depressurization portion 213. In particular, to achieve a high degree of flexibility that the bending portion 202 needs to have, a material having a low bending rigidity is used in this portion. If the depressurization portion 213 and the outer tube 212 disposed in the bending portion 202 are both made of resin materials, the JT cooling apparatus 210 can have sufficient flexibility in the portion of the endoscope 102 that needs to have the highest bendability. Although it is desirable that the flexible depressurization portion 213 extends along the entire length of the bending portion 202 as described above, the depressurization portion 213 may extends along a part of the bending portion 202, as long as it extends in the bending portion of the endoscope and having the function of depressurization. As described later, the depressurization portion 213 is provided in order to change the refrigerant gas at a normal temperature and a high pressure into low temperature, low pressure gas as needed. To achieve this, for example, the length, inner diameter, and material of the depressurization portion 213 is suitably selected.

The refrigerant gas having an appropriate pressure adjusted by the refrigerant gas controller 111 (see FIG. 1) flows into the channel 216 inside the inner tube 214 of the heat exchanger 215. As the refrigerant gas flows in this channel 216, the gas is precooled by the low temperature, low pressure refrigerant gas after expansion flowing in the channel 217 between the inner tube 214 and the outer tube 212. In the JT cooling apparatus 215, since the Joule-Thomson coefficient (i.e. a change in the temperature per unit change in the pressure) generally varies greatly depending on the temperature, it is important, in order to lower the temperature of the refrigerant gas efficiently, to precool the normal temperature, high pressure refrigerant gas before depressurizing it in the depressurization portion 213.

The refrigerant gas precooled by the heat exchanger 215 then flows into the depressurization portion 213. Since the depressurization portion 213 is a very thin tube (or a very thin tube) having an inner diameter smaller than that of the inner tube 214, the pressure of the high pressure refrigerant gas falls by a large loss of gas pressure, and consequently the temperature of the refrigerant gas also falls by the Joule-Thomson effect. The temperature and pressure of the refrigerant gas change from a normal temperature and high pressure to a low temperature and low pressure through the depressurization portion 213. Then in the end chip 211, the refrigerant gas exchanges heat with the end chip 211, namely, the refrigerant gas absorbs heat from the end chip 211, and therefore absorbs heat from the image pickup module 220, which is the object to be cooled. Then, the refrigerant gas flows into the channel 217.

Here, “low pressure” means a pressure lower than the pressure in the initial high pressure state, and the “low pressure” is equal to or close to the atmospheric pressure. At the time when the refrigerant gas flows into the channel 217, the temperature thereof has not returned to a normal temperature in typical cases. Therefore, as the refrigerant gas flows in the channel 217, it precools the normal temperature, high pressure refrigerant gas flowing in the channel 216 in the heat exchanger 215, as described above. As the refrigerant gas passes through the channel 217, the temperature becomes close to a normal temperature, and thereafter it is discharged into the environment.

As the refrigerant gas, use may be made of any gas having a positive Joule-Thomson coefficient in the temperature range in which it is used, namely any gas whose temperature falls with a fall of its pressure. Specifically, the refrigerant gas may be, for example, air, nitrogen, argon, carbon dioxide, or dinitrogen monoxide (N₂O). Among them, carbon dioxide and dinitrogen monoxide are particularly preferred, because it is desirable that the refrigerant gas has a large Joule-Thomson coefficient and does not have flammability nor toxicity. A mixture gas having a large Joule-Thomson coefficient may also be used.

As described in the foregoing, with the use of the JT cooling apparatus 210 having a long, substantially concentric double tube structure according to the first embodiment, low temperature can be achieved in the neighborhood of the object to be cooled while achieving required flexibility of the endoscope. In particular, even in the case where an object to be cooled is in the end portion of a thin long structure like an endoscope having an insertion portion with a length larger than 1 meter, the object can be cooled to a temperature lower than the environmental temperature.

### Second Embodiment

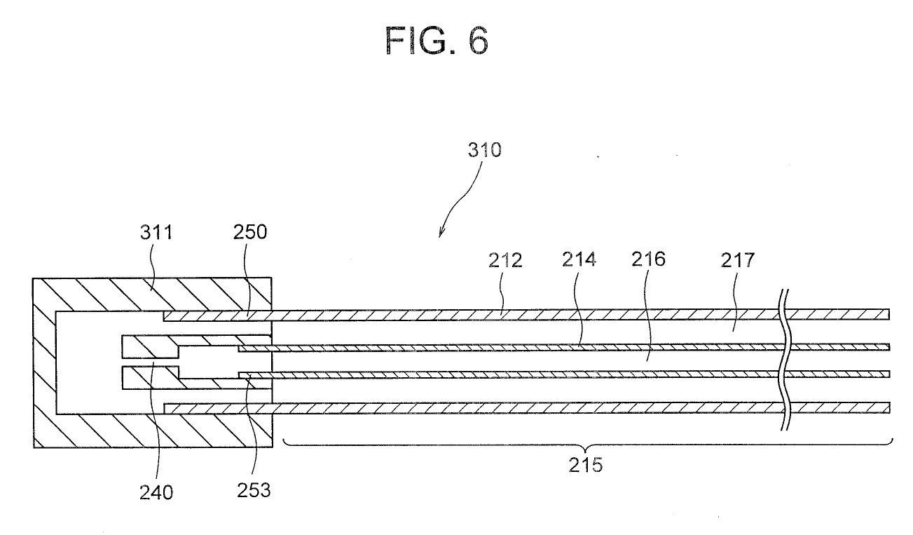

In the following, an endoscope equipped with a Joule-Thomson cooling apparatus having a depressurization portion disposed in the end chip according to a second embodiment will be described. FIG. 6 is a cross sectional view of the Joule-Thomson cooling apparatus 310 having a depressurization portion 240 disposed in the end chip 311 according to the second embodiment. An inner tube 214 is a single tube, which is connected airtightly with the end chip 311 by a connection portion 253. An outer tube 212 is connected airtightly with the end chip 311 by a connection portion 250. Refrigerant gas at a normal temperature and a high pressure introduced into a channel 216 inside the inner tube 214 is precooled by a heat exchanger 215, and then depressurized in the depressurization portion 240 in the end chip 311 to become low temperature, low pressure gas, which issues from the depressurization portion 240. The low temperature, low pressure refrigerant gas issuing from the depressurization portion 240 exchanges heat in the end chip 311, then precools the refrigerant gas flowing in the channel 216 as it flows in the channel 217, and is discharged to the exterior.

In order for the bending portion 202 (see FIG. 2) of the endoscope to have sufficient bendability, and in order to achieve overall heat insulation of the JT cooling apparatus 310, it is preferred that the outer tube 212 and the inner tube 214 be both made of a resin material. It is necessary for the depressurization portion 240 to cause a pressure fall substantially equal to that in the depressurization portion 213 in the first embodiment shown in FIG. 5, across a distance shorter than that in the first embodiment. Therefore, the diameter of the channel in the depressurization portion 240 is designed to be much smaller than that in the depressurization portion 213.

In this structure, the depressurization portion 240 is produced in the end chip 311 by machining or MEMS processing. Therefore, it can be produced advantageously with a higher accuracy as compared to the case of a tube with a very small diameter produced by drawing.

In the above description of the second embodiment, only the elements different from those in the first embodiment have been described, and the elements that have not described are the same as those in the first embodiment. For example, although the endoscope equipped with the JT cooling apparatus 310 according to the second embodiment and the endoscope system are not illustrated in the drawing, the JT cooling apparatus 310 according to the second embodiment can be used in a manner similar to the JT cooling apparatus 210 of the endoscope 102 according to the first embodiment.

Although the endoscopes according to the first and second embodiments are flexible endoscopes having a bending portion, the present invention can also be applied to rigid endoscopes.

In the endoscope according to the present invention, a JT cooling apparatus that cools an object to be cooled is provided in the vicinity of the object such as an electronic component. Therefore, it is possible to cool the object to a temperature lower than the environmental temperature without being restricted by the environmental temperature.

What is claimed is:

1. An endoscope comprising:
an insertion portion;
an electronic component housed in a distal end portion of the insertion portion;
a Joule-Thomson cooling apparatus provided in a tube of the endoscope to cool the electronic component.

2. The endoscope according to claim 1, wherein the Joule-Thomson cooling apparatus comprises a straight double tube.

3. The endoscope according to claim 2, wherein the double tube is flexible.

4. The endoscope according to claim 3, wherein the Joule-Thomson cooling apparatus comprises a very thin tube that extends at least in a bending portion of the endoscope and functions as a depressurization portion.

5. The endoscope according to claim 4, wherein the length of the depressurization portion is longer than the bending portion of the endoscope, the depressurization portion extends along the entire length of the bending portion of the endoscope portion, and the depressurization portion is made of a resin material.

6. The endoscope according to claim 3, wherein an inner tube and an outer tube that constitute the double tube are both made of a resin material, and the depressurization portion is located in an end chip.

7. The endoscope according to claim 2, wherein the Joule-Thomson cooling apparatus comprises a very thin tube that extends at least in a bending portion of the endoscope and functions as a depressurization portion.

8. The endoscope according to claim 7, wherein the length of the depressurization portion is longer than the bending portion of the endoscope, the depressurization portion extends along the entire length of the bending portion of the endoscope portion, and the depressurization portion is made of a resin material.

9. The endoscope according to claim 1, wherein the Joule-Thomson cooling apparatus comprises a very thin tube that extends at least in a bending portion of the endoscope and functions as a depressurization portion.

10. The endoscope according to claim 9, wherein the length of the depressurization portion is longer than the bending portion of the endoscope, the depressurization portion extends along the entire length of the bending portion of the endoscope portion, and the depressurization portion is made of a resin material.
